(19) United States
(12) Patent Application Publication
(10) Pub. No.: US 2024/0327903 A1
(43) Pub. Date: Oct. 3, 2024

(54) Method for Placing a Partition Cohort of a Microfluidic, in Particular Biological Sample

(71) Applicant: Robert Bosch GmbH, Stuttgart (DE)

(72) Inventors: Manuel Loskyll, Reutlingen (DE); Daniel Sebastian Podbiel, Rutesheim (DE)

(21) Appl. No.: 18/574,114

(22) PCT Filed: Jun. 3, 2022
(86) PCT No.: PCT/EP2022/065161

§ 371 Date: Jan. 29, 2024

(30) Foreign Application Priority Data
Jun. 29, 2021 (DE) 10 2021 206 717.4

(51) Int. Cl. C12Q 1/6851; C12Q 1/6806

(52) U.S. Cl. CPC C12Q 1/6851; C12Q 1/6806

(57) ABSTRACT

A method is for placing a partition cohort of a microfluidic, in particular biological sample, in partitions on a partition surface, in particular to determine a concentration of an analyte in the sample. The method includes establishing a geometric shape of the partitions, a shape of the partition surface, and a total volume of the sample, and determining a minimum partition dimension of the partitions and a corresponding maximum number of the partitions that can be arranged on the partition surface and have a minimum partition dimension as a first partition number. The method further includes ascertaining a second partition dimension of the partitions and a corresponding second partition number under the condition that a maximum possible proportion of the total volume can be partitioned on the partition surface, and the maximum possible proportion is divided on as many partitions as possible on the partition surface.

### THE PRIOR ART

Microfluidic analysis systems (referred to as lab-on-chips or LOCs) enable automated, reliable, fast, compact, and cost-effective processing of patient samples for medical diagnostics. By combining a variety of operations for controlled manipulation of fluids, complex molecular diagnostic test procedures can be performed on a lab-on-chip cartridge.

Given the implementation of a partition-based quantitative test method, particularly in what is referred to as digital polymerase chain reaction (abbreviated as digital PCR) into such a LoC, one of these operations consists of aliquoting the sample into a plurality of partitions. These can, e.g., be realized as chambers or droplets. Within each of these partitions, an end point amplification of the specific DNA target takes place independently of each other, which results in an increase in fluorescence if at least one target was present in the respective partition at the start of the amplification reaction. Based on the proportion of partitions that exhibit an increase in fluorescence, the target concentration in the analyzed sample can be inferred by using Poisson statistics.

During the aliquoting process, various sources of error affect the accuracy of the quantitative detection. In this context, the fluctuations in the concentration of the examined partial volume (referred to as the subsampling error) and the statistical inaccuracy due to the aliquoting process itself (referred to as the partitioning error) are dominant. Both of these mechanisms have previously been described in the literature (Dube et al., PLoS ONE 3 (8), e2876 (2008), 10.1371/journal.pone.0002876; Jacobs et al., BMC Bioinformatics 15, 283 (2014), https://doi.org/10.1186/1471-2105-15-283; Bizouarn F. (2014) Introduction to Digital PCR. In: Biassoni R., Raso A. (eds) Quantitative Real-Time PCR. Methods in Molecular Biology (Methods and Protocols), vol 1160. Humana Press, New York, NY. https://doi.org/10.1007/978-1-4939-0733-5_4; Basu, Amar S. (2017): Digital Assays Part I: Partitioning Statistics and Digital PCR. in SLAS technology 22 (4), pp. 369-386. DOI: 10.1177/2472630317705680). The general recommendation thereby is to reduce the partitioning error by using the largest possible number of partitions in order to thereby increase the dynamic measurement range of the analysis system. The subsampling error is generally negligible and, according to the literature, only plays a significant role if the proportion of the analyzed volume is below 30% or 50%, depending on the application (Jacobs et al., BMC Bioinformatics 15, 283 (2014), https://doi.org/10.1186/1471-2105-15-283). In addition, the advantage of as many partitions as possible is emphasized.

### DISCLOSURE OF THE INVENTION

### Advantages of the Invention

Against this background, the invention relates to a method for placing a partition cohort of a microfluidic sample in partitions on a partition surface. In particular, the invention further relates to a method for partitioning, dividing, or aliquoting such a sample into partitions or sub-quantities of the sample. The method can in particular be used for determination of a concentration of an analyte in the sample, in particular in combination with digital PCR in the case of nucleic acid sections as analytes.

A partition cohort of a sample can therefore be understood as dividing or aliquoting of a sample into sample portions as described and, in a preferred embodiment, an actual placement of the sample in the form of sample portions on the partition surface. In particular, the sample can be a biological sample, e.g. comprising a body fluid, e.g. blood, urine or sputum, or a swab. The sample can contain other substances in addition to the body fluid, e.g. in the form of a mixture of the body fluid with a transport medium such as eNAT™ or UTM™. The analyte, also referred to as the target, can be a molecule in the sample, in particular a nucleic acid portion, e.g. a part of a DNA or RNA of a pathogen or a particular body cell, in particular a tumor cell. In one particular embodiment, the analyte can also be cells, in particular single or animal or human cells, e.g., one or a plurality of what are referred to as circulating tumor cells (CTC). As previously mentioned, the method according to the invention can therefore be part of or a prerequisite for a partition-based quantitative detection method for analytes in the sample, e.g. with the aid of a digital PCR, whereby such a detection method for microfluidic samples can preferably be performed using a microfluidic device, in particular a lab-on-chip system. Using the method according to the invention, a partition cohort of the sample for a statistical estimation of a concentration of the analyte in the sample can advantageously be achieved at a low relative error.

In one preferred embodiment, the method for placing a partition cohort comprises a partition cohort of the sample on the partition surface according to the present invention. The partition surface can generally be part of a microfluidic device, preferably part of a microfluidic cartridge. For example, the partition surface can be a surface on a substrate in a chamber of the microfluidic device.

A first method step establishes a geometrical shape of the partitions, a shape of the partition surface, and a total volume of the sample. The partition surface can, e.g., have a square or rectangular shape and can optionally be surrounded by a limiting wall. The partitions can preferably be in the form of droplets or chambers. In the case of the droplets, these are present in particular as an emulsion, e.g. as water-based droplets in an oil emulsion, whereby the droplets can preferably be stabilized by the addition of surfactants. In the case of chambers, the chambers can have a round or square base surface, preferably a hexagonal base surface. The chambers are in this case separated from each other by walls, the walls preferably being designed as thin as possible in order to block as little area as possible. Preferably, the walls are taken into consideration regarding the geometrical shape of the chambers.

In a further method step, a minimum partition dimension of the partitions is determined. A minimum partition dimension is understood to mean a measure, i.e., a representative and preferably an unambiguous value for the size of the partition, in particular for the volume of the partition. In particular, the partition dimension can be the diameter of the partition for a spherical or droplet shaped partition, or a width of the chamber for a partition cohort in chambers with an angular, e.g. square or hexagonal, base surface. For example, the partition dimension is the distance between two opposite corners, preferably the minimum midpoint distance of the partitions. The minimum partition dimension implicitly results in a maximum number of partitions that can be arranged on the partition surface with a minimum partition dimension. This maximum number is also hereinafter referred to as the first partition number and is a measure of a minimum value of the partitioning error, because as the number of partitions increases, a statistical inaccuracy of the concentration determination of the total partitioned volume of the sample is reduced. Placing a partition cohort having as many partitions as possible and correspondingly small partition dimension is referred to hereinafter as a minimum variant.

According to a further method step, a second partition dimension of the partitions, and thus a second partition number, is ascertained under the condition that a maximum possible proportion of the total volume can be partitioned on the partition surface and, given this requirement, the maximum possible proportion is divided over as many partitions as possible on the partition surface. This has the advantage that the more of the total volume can be partitioned, the lower the subsampling error will be. However, the number of partitions should in this case still be kept as large as possible in order to achieve the lowest possible partitioning error. This second partition dimension is also hereinafter referred to as the maximum partition dimension dₘₐₓ, but represents the smallest partition dimension possible for a maximum partitioned total volume. The number of partitions for the maximum partition dimension is also referred to hereinafter as the second partition number Aₘᵢₙ. A placement of a partition cohort in which the complete total volume of the sample is partitioned as far as possible and a larger partition dimension is selected accordingly is referred to hereinafter as the maximum variant in order to distinguish it from the minimum variant. This step can in particular also be performed before the previous step.

In a subsequent method step, a first uncertainty of a measurable concentration of the analyte is determined using a partition dimension from a first area around the minimum partition dimension, and a second uncertainty of the measurable concentration is determined using a partition dimension from a second area around the second partition dimension. The first and the second uncertainties can preferably be relative uncertainties. These determinations advantageously ascertain whether a placement according to the minimum or maximum variant is superior with respect to the lowest possible measurement error of the concentration. The first area is preferably defined such that the first area comprises the minimum partition dimension, preferably as a lower end of the first area, and does not comprise the second partition dimension. The second area is preferably defined such that the second area comprises the second partition dimension, preferably as an upper end of the second area, and does not comprise the minimum partition dimension. In one particular embodiment, the first area and the second area can be two mutually non-overlapping areas, i.e. in particular disjunctive areas.

According to a particular embodiment of this method step, the first uncertainty of a measurable concentration of the analyte is determined using the first partition number of partitions having the minimum partition dimension, and the second uncertainty of the measurable concentration is determined using the second partition number of partitions having the second partition dimension. Preferably, the first uncertainty and/or the second uncertainty are determined at a value based on the maximum theoretical area for a measurement of the concentration of the analyte. This maximum theoretical area extends from −log(1−1/Aₘₐₓ)/Vₚₐᵣ to −log(1−(Aₘₐₓ−1)/Aₘₐₓ)/Vₚₐᵣ where Aₘₐₓ denotes the first partition number, Vₚₐᵣ denotes the capacity/volume of a single partition and log denotes the natural log. In order to preferably generate two equally large measurement areas, the value of the midpoint of this area is selected on a logarithmic scale, which is (log(1−1/Aₘₐₓ)*log(1−(Aₘₐₓ−1)/Aₘₐₓ))\hat{ }0.5/Vₚₐᵣ.

According to a particular embodiment of the method, the partitions having a partition dimension from the first area, preferably having the minimum partition dimension, are placed when it is not possible to measure a concentration of the analyte at a specified concentration value when the partitions having a partition dimension based on the second area, in particular having the second partition dimension, are placed. The specified concentration value can preferably be a value based on the maximum theoretical area described hereinabove, preferably the midpoint of that area described hereinabove on a logarithmic scale.

In a subsequent method step, the partitions are placed from the first area or the second area, depending on a comparison of the first uncertainty with the second uncertainty. This has the advantage that uncertainties for a subsequent concentration measurement, and thus for the specific shape of the placement of the partitions, can be taken into account when selecting the partition dimension. In particular, a choice of the minimum variant or maximum variant can be made based on these ascertained uncertainties.

In a particular embodiment of the method, the partitions having a partition dimension unequal to the maximum partition dimension are placed when the first uncertainty is less than the second uncertainty, and the partitions unequal to the minimum partition dimension are placed when the first uncertainty is greater than or equal to the second uncertainty. In this embodiment, the first area therefore comprises all achievable partition dimensions except for the second/maximum partition dimension, so the second area comprises all achievable partition dimensions except for the minimum partition dimension.

In a further particular embodiment of the method, a partition dimension from the first area is placed when the first uncertainty is less than the second uncertainty, and the partitions having a partition dimension from the second area are placed when the first uncertainty is greater than or equal to the second uncertainty. In this case, an area (optionally modified from the previous method step) can be selected as the first area such that the area only comprises partition dimensions whose associated, preferably relative, uncertainty of the measurable concentration is less than the preferably relative uncertainty of the measurable concentration when the second/maximum partition dimension is selected. Similarly, an area (optionally modified from the previous step) can be selected as the second area such that the area only comprises partition dimensions whose associated, preferably relative, uncertainty of the measurable concentration is less than the preferably relative uncertainty of the measurable concentration when the minimum partition dimension is selected. This has the advantage that a partition dimension of the minimum variant is selected, for which the relative uncertainty is always smaller than when the maximum variant is selected, and vice versa.

In a further particular embodiment of the method, the partitions acting as the first partition number of partitions having the minimum partition dimension are placed when the first uncertainty is less than the second uncertainty, or acting as the second partition number of partitions having the second/maximum partition dimension are placed when the first uncertainty is greater than or equal the second uncertainty.

As explained hereinabove, the method can be part of an analysis method for detecting an analyte in a microfluidic sample. The object of the invention is therefore also such an analysis method, whereby the sample is divided into partitions according to the invention and whereby at least one partition is examined for the presence of the analyte. The analysis method in this case preferably comprises performing a digital PCR on at least some of the partitions.

The object of the invention is also a method for preparing a partition cohort of a microfluidic sample. A partition surface is provided thereby, and a partition cohort of the sample is performed according to the placement method according to the invention. The preparation method can preferably comprise the preparation of chambers for the partition cohort on the partition surface.

### BRIEF DESCRIPTION OF THE DRAWINGS

Exemplary embodiments of the invention are shown schematically in the drawings and explained in more detail in the following description. Identical reference signs are used for elements shown in the drawings and having identical functions, so repeated description of these elements is omitted.

### Shown are:

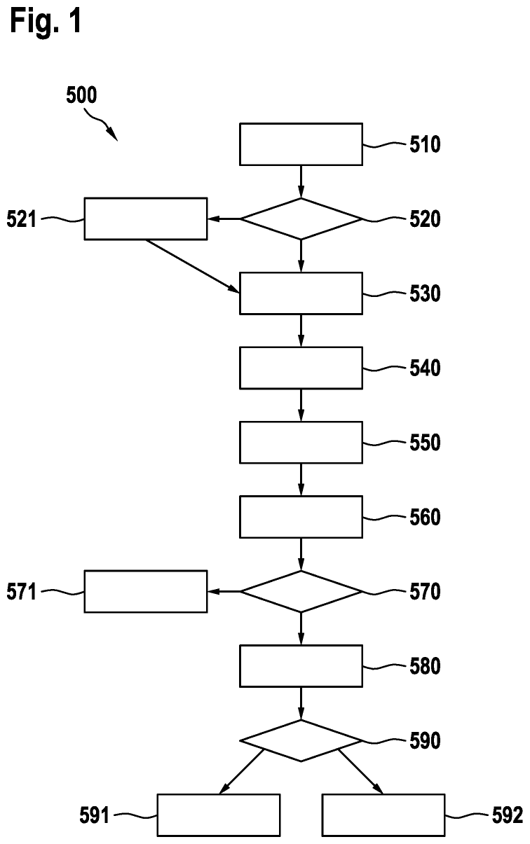

FIG. 1 a flow chart of an exemplary embodiment of the method according to the invention for placing a partition cohort,

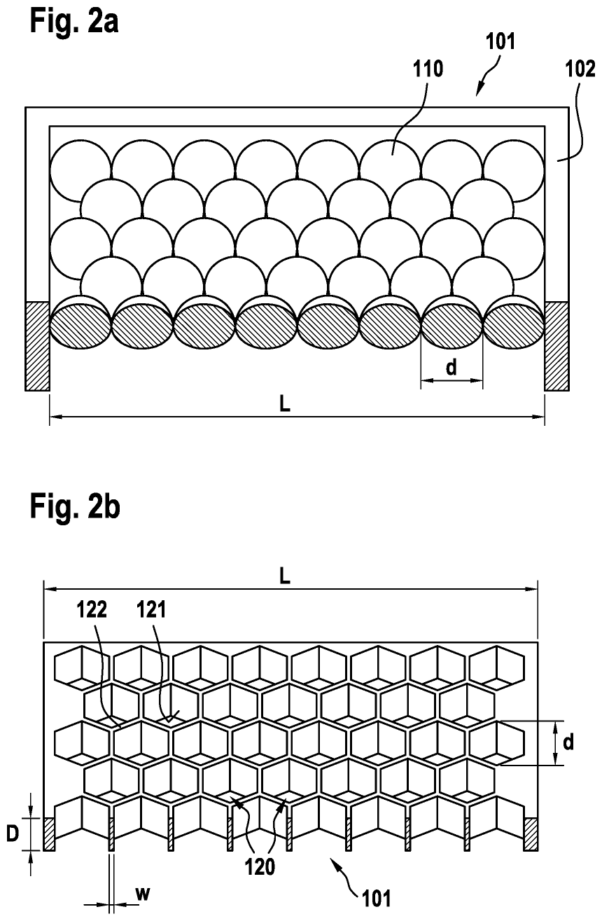

FIGS. 2a, 2b a schematic representation of a partition cohort of a sample on a partition surface in the form of droplets or in the form of chambers filled with sample parts,

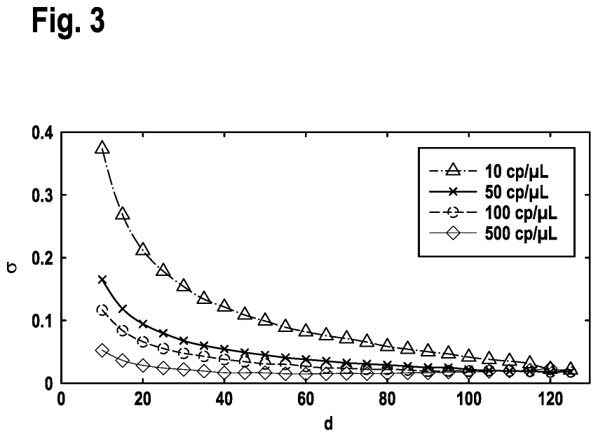

FIG. 3 diagrams with exemplary calculations of relative uncertainties of various concentrations of an analyte in the sample depending on a selected partition dimension for the partitions,

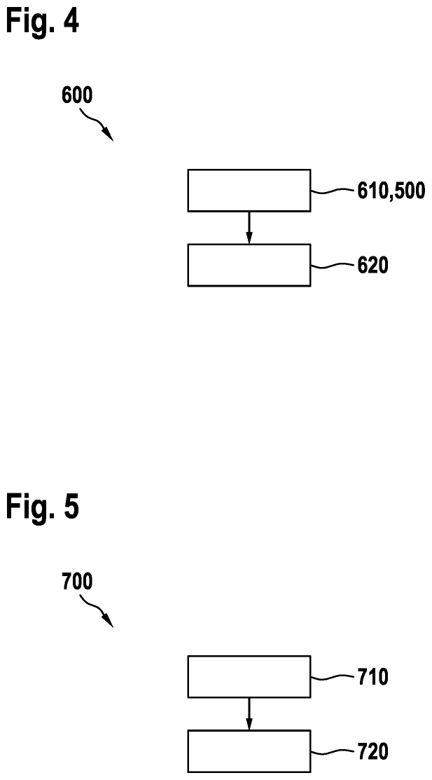

FIG. 4 a flow chart of an exemplary embodiment of the analysis method according to the invention for detecting an analyte in a microfluidic sample, and

FIG. 5 a flow chart of an exemplary embodiment of the preparation method according to the invention of a partition cohort of a microfluidic sample.

### EMBODIMENTS OF THE INVENTION

FIG. 1 shows a flow chart 500 of an exemplary embodiment of the method 500 according to the invention for placing a partition cohort of a microfluidic sample. In this case, as described hereinabove, the partition cohort can in particular be part of or a prerequisite for a partition-based quantitative detection method for analytes in the sample, whereby such a detection method for microfluidic samples can preferably be performed using a microfluidic device, in particular a lab-on-chip system. For example, the method described hereinafter is part of a quantitative detection of nucleic acid portions of disease agents or tumor cells in a biological sample comprising a body fluid (e.g., blood, sputum, or a smear), whereby the quantitative detection is performed using a digital PCR with the partition cohort of the sample designed according to the present invention.

In a first step 510 of method 500, the volume of the sample and an area available for the partition cohort, or partition surface, are determined. By way of example, the dividable volume V_{sys}=25 microliters (μL) and the partition surface could be designed in the shape of a square with an edge length of L=10 millimeters (mm).

In the second step 520, which can also be performed before the first step 510, it is established whether the partition cohort of the sample is divided in the form of droplets without intermediate walls or in chambers. FIG. 2a schematically shows an arrangement of droplets 110 with diameter d on a partition surface 101 having a width L, whereby the partition surface 101 can, e.g., be square or rectangular and limited by a wall 102, as shown. The partition surface 101 can in this case in particular be arranged in a chamber of a microfluidic cartridge. FIG. 2b schematically shows, in an alternative embodiment, an arrangement of chambers 120 on the partition surface 101, whereby the sample is to be divided into the chambers 120. As illustrated, the chambers 120 have, e.g., a hexagonal base surface 121 having a width d, a wall height D that corresponds to a chamber depth, and a wall thickness w between two chambers, whereby the wall thickness w is preferably selected as small as possible to block as little area of the partition surface 101 as possible by walls 122 of the chambers 120. A minimum wall thickness and maximum wall height to maximize the volume of the chambers 120 may in this case in particular also be limited by manufacturing requirements. When the chamber-based arrangement 120 is selected, a sub-step 521 establishes the minimum wall thickness w, e.g. w=25 micrometers (μm), and the wall height/chamber depth D, e.g. D=380 μm. The partition surface 101 can in particular be a surface of a substrate, e.g., a substrate of silicon, plastic, or a combination of both materials. In the case of the embodiment with chambers 120, the chambers 120, in particular the walls 122, can also be made of silicon, plastic, or a combination of both materials. In the case of an aqueous sample in particular, a hydroplethilic design of the partition surface 101 or the chambers 120 is advantageous.

Subsequently, in a third step 530, a minimum partition dimension dₘᵢₙ is defined, whereby a size of this minimum partition dimension dₘᵢₙ can be determined by, depending on the individual case, the aforementioned manufacturing limitations and a resolution of detection optics to be used for the analysis of the partitions. For example, the minimum partition dimension is dₘᵢₙ=10 μm and represents, preferably corresponds to, the diameter d of the droplet 110 in the case of the droplet-based arrangement or the distance between the midpoints of two chambers 120 or the distance d between two opposite corners in the case of a hexagonal base surface 121 of the chambers 120 in the case of the chamber-based arrangement. Based on this minimum partition dimension dₘᵢₙ, a fourth step 540 calculates how many partitions can be accommodated on the partition surface 101, optionally while maintaining the minimum wall thickness w. In the case considered, with a chamber-based approach, this would be Aₘₐₓ=101871 partitions in hexagonal arrangement, whereby this number is hereinafter referred to as the first partition number Aₘₐₓ. The dimensions of the partitions together with the depth D of the chambers 120 can be used to calculate the capacity of a single partition Vₚₐᵣ and, after multiplication by the first partition number Aₘₐₓ, the total tangible volume Vₐₙₐ. In the present case, this is about Vₐₙₐ=2.51 μL and is therefore only about one tenth of the total dividable volume V_{sys} of 25 μL.

The first partition number Aₘₐₓ also defines the maximum theoretical area for a measurement of the concentration of the analyte (with cp/μL as the unit for copies per microliter) ranging from −log(1−1/Aₘₐₓ)/Vₚₐᵣ to −log(1−(Aₘₐₓ−1)/Aₘₐₓ)/Vₚₐᵣ, where log denotes the natural logarithm. In order to preferably generate two equally large measurement ranges, this maximum measurement range is halved on a logarithmic scale according to a fifth step 550. The resulting midpoint k is then (log(1−1/Aₘₐₓ)*log(1−(Aₘₐₓ−1)/Aₘₐₓ))\hat{ }0.5/Vₚₐᵣ and in this example corresponds to about 431 cp/μL. Alternatively, a different concentration value k can also be selected from this measurement range, particularly if there are reasons for a preferred measurement at lower or higher concentrations.

A preferred alternative to as many partitions as possible is to choose the dimensions of the partitions so that as much of the total volume of V_{S}s as possible is also partitioned. If the partition surface and a height filled by the partitions are large enough, it is always possible to accommodate the total volume of V_{sys} on the partition surface 101, in the limiting case in just one single partition. However, in order to also keep the partitioning error as low as possible, the volume should be divided into as many partitions as possible when using the maximum possible volume. According to the sixth step 560 of method 500, a second partition dimension dₘₐₓ must first be found, which, under the given conditions, maximizes the partitionable and thus analyzable volume Vₐₙₐ and at the same time implies as many partitions as possible. This second partition dimension is also referred to as the maximum partition dimension dₘₐₓ, but represents the smallest partition dimension possible for a maximum partitioned total volume. In the illustrated example, dₘₐₓ is 128.52 μm with a maximum partitionable volume Vₐₙₐ of 24.999 μL, i.e., practically the total volume V_{sys} of 25 μL. The partitionable volume of Vₐₙₐ is thereby divided into 6132 partitions, whereby this number is hereinafter referred to as the second partition number Aₘᵢₙ. The sixth step 560 can also be performed earlier, in particular before or parallel to any of the preceding steps, provided the first step 510 and the second step 520 have been performed.

According to a preferable seventh step 570 of the method, the maximum theoretical area for a measurement of the concentration of the analyte is ascertained when using the second partition number Aₘᵢₙ of partitions with maximum partition dimension dₘₐₓ, in which case this area extends from −log(1−1/Aₘᵢₙ)/Vₚₐr₂ to −log(1−(Aₘᵢₙ−1)/Aₘᵢₙ)/Vₚₐr₂, similar to hereinabove, and where Vpar_2 corresponds to the volume of a partition dimension dₘₐₓ. If the midpoint λ calculated hereinabove does not fall within this area, preferably the partition cohort is placed according to a sub-step 571 of the seventh step 570 according to the first partition number Aₘₐₓ with minimum partition dimension dₘᵢₙ. If the calculated midpoint λ falls within this area, then the method 500 proceeds according to this exemplary embodiment as follows.

According to an eighth step 580 of method 500, a first uncertainty of a measurable concentration of the analyte is calculated using the first partition number Aₘₐₓ of partitions with minimum partition dimension dₘᵢₙ (hereinafter referred to as minimum variant) and a second uncertainty of the measurable concentration is calculated using the second partition number Aₘᵢₙ of partitions with maximum partition dimension dₘₐₓ (hereinafter referred to as maximum variant).

The relative uncertainties can in this case preferably be determined using the following approach.

In order to calculate the partitioning error, an uncertainty can be used based on the confidence interval of the following probability distribution P(C|H):

$$P ( C \vert H ) = \frac{( A - 1 ) !}{( A - A - 1 ) !} \cdot \frac{{S}_{C , H}}{{A}^{C}}$$

where C is the number of targets in the analyzed sample volume Vₐₙₐ, H is the number of partitions containing the at least one analyte, A is the available number of partitions, and S is the second type Stirling number.

Regarding the subsampling error, an uncertainty can be used based on the confidence interval of the following probability distribution P(Cₜₒₜ|C, p):

$$P ( {C}_{tot} \vert C , p ) = ( \begin{matrix}{C}_{tot} \\ C\end{matrix} ) \cdot {p}^{C + 1} \cdot {( 1 - p )}^{{C}_{tot} - C}$$

where C is the number of targets in the analyzable sample volume Vₐₙₐ, Cₜₒₜ is the number of targets in the partitionable volume V_{sys} and p is the proportion of the analyzable volume in the partitionable volume Vₐₙₐ/V_{sys}.

If both errors are combined, the result is:

$$P ( {C}_{tot} \vert H , p ) = {\sum }_{C = H}^{{C}_{tot}} P ( {C}_{tot} \vert C , p ) \cdot P ( C \vert H )$$

Given that an exact calculation of this distribution takes very long, it can be approximated by a Gaussian distribution. Given a Gaussian distribution, the confidence interval can be calculated directly from the variance s. In the case of a 95% confidence interval, this is μ±1.96·\sqrt{s}, where μ represents the expected value. The standard deviation \sqrt{s} of a Gaussian distribution can be calculated from the probability at the expected value, i.e. the maximum probability:

$$\sqrt{s} = \frac{1}{\sqrt{2 π} \cdot P ( μ )}$$

This relationship is used in the approximation. The maximum value of P(Cₜₒₜ|H, p) is set equal to P(μ), before the standard deviation and the confidence interval are calculated, which in turn defines the inaccuracy.

The value at which P(Cₜₒₜ|H, p) is maximum is:

$${\hat{C}}_{tot} = - ⌊ - log ( 1 - \frac{H}{N} ) \cdot \frac{N}{p} ⌋$$

The calculation of P(Cₜₒₜ|H, p) is performed as follows:

$$P ( {\hat{C}}_{tot} \vert H , p ) = \frac{( N - 1 ) !}{( N - H - 1 ) !} {\sum }_{C = H}^{{\hat{C}}_{tot}} \frac{{S}_{C , H}}{{N}^{C}} \cdot ( \begin{matrix}{\hat{C}}_{tot} \\ C\end{matrix} ) \cdot {p}^{C + 1} \cdot {( 1 - p )}^{{\hat{C}}_{tot} - C}$$

This calculation is advantageous with regard to the time expenditure. This approximation enables a qualitative comparison, and the quantitative results are in the range of about 10% around the true, exact value of uncertainty.

Using this approximation in regard to the present example, the first relative uncertainty of the measured result a at the midpoint λ is about 0.0566 or 5.66% when using the minimum variant. When the maximum variant is used, the second relative uncertainty at the midpoint λ is about 0.0254 or 2.54%, i.e., less than half as much the relative uncertainty

According to a ninth step 590 of method 500, the two relative uncertainties are now compared and, depending on this comparison, a placement of the partition cohort is determined. In particular, depending on this comparison, a partition cohort according to the minimum variant 591 or according to the maximum variant 592 can be achieved such that preferably the relative uncertainty is as low as possible. Regarding the specific choice of the variant, and in particular for the choice of a specific value for the partition, several approaches, which were in particular explained hereinabove, are advantageous. For example, the placement which provides the lower uncertainty in the middle of the maximum theoretical measuring range can then be selected. In this example, with a square partition surface of 10 mm edge length, a processed volume of 25 L, a chamber depth/wall height of 380 μm, a minimum wall thickness of 25 μm and minimum partition dimension of 10 μm, the sample is distributed into 6132 hexagonal partitions with a partition dimension of 128.52 μm. After filling the chambers 120 with the sample according to this partition cohort, in a particular embodiment the chambers 120 can be coated with (transparent) oil before subsequent processing or analysis, e.g. as part of a digital PCR.

FIG. 3 shows, by way of example, relative uncertainties a calculated according to the approximation described hereinabove as a function of the partition dimension d for different concentrations of analyte in the sample (10, 50, 100, and 500 analytes per microliter, respectively) using chambers 120 and the same values for volume, edge length, chamber depth/wall height and wall thickness. As can be seen from FIG. 3, for these boundary conditions, a placement with large partition dimensions is usually associated with a lower relative error for all calculated concentrations, whereby a placement with the smallest possible partition dimension is unfavorable, particularly for low concentrations. By way of explanation, in these cases the subsampling error dominates over the partitioning error due to the significantly smaller partitionable volume with a very small partition dimension. For other boundary conditions, however, smaller relative errors can also result for certain concentrations for placements with a small partition dimension compared to placements according to the maximum variant.

According to a modification of the exemplary embodiment 500 described hereinabove, the total dividable volume V_{sys} is increased to 30 μL. The minimum partition dimension dₘᵢₙ, the edge length L of the partition surface 101, the wall height/chamber depth D and the wall thickness w remain unchanged at 10 μm, 10 mm, 380 μm and 25 μm, respectively. In this case, the midpoint λ of the logarithmic measurement range is about 431 cp/μL. The largest proportion of the dividable volume of V_{sys} can be analyzed for a second/maximum partition dimension of dₘₐₓ=294.65 μm. For this partition dimension, the measurement range reaches a concentration of approximately 338 cp/μL. Accordingly, this measurement range does not include the midpoint λ, so the partitioning would preferably be placed in accordance with sub-step 571 of the seventh step 570 according to the minimum variant.

FIG. 4 shows a flow chart of an exemplary embodiment of the analysis method 600 according to the invention. In a first step 610 of method 600, a sample can be preferably divided into partitions according to the method of the invention for placing a partition cohort in partitions, e.g. according to method 500 described hereinabove with respect to FIG. 1. Subsequently, in a second step 620, at least one partition, preferably all partitions, can be examined for the presence of the analyte and a concentration of the analyte in the divided sample can be inferred on this basis. This determination of the concentration is in this case preferably performed via the performance of a digital PCR, for which the partitions form the basis.

FIG. 5 shows a flow chart of an exemplary embodiment of the preparation method 700 according to the invention. In a first step 710, a partition surface is provided. This can, e.g., be performed by providing a substrate, whereby one surface of the substrate forms the partition surface. The partition surface or substrate can in this case be part of a microfluidic cartridge or can be arranged or fixed in the cartridge in the course of the preparation method 700. In a second step 720 of the method, the partition cohort is placed according to the method according to the invention for placing a partition cohort, e.g. according to the method 500 described hereinabove with respect to FIG. 1. In this case, one advantageous embodiment of the preparation method 700 can comprise a preparation of chambers for the partition cohort on the partition surface.

1. A method for placing a partition cohort of a microfluidic sample in partitions on a partition surface to determine a concentration of an analyte in the microfluidic sample, the method comprising:
establishing a geometric shape of the partitions, a shape of the partition surface, and a total volume of the microfluidic sample;
determining a minimum partition dimension of the partitions and a corresponding maximum number of the partitions that can be arranged on the partition surface and have a minimum partition dimension as a first partition number;
ascertaining a second partition dimension of the partitions and a corresponding second partition number under a condition that a maximum possible proportion of the total volume is partitioned on the partition surface, and the maximum possible proportion is divided on as many partitions as possible on the partition surface;
determining a first uncertainty of a measurable concentration of the analyte using a partition dimension from a first area around the minimum partition dimension, and determining a second uncertainty of the measurable concentration using a partition dimension from a second area around the second partition dimension; and
placing the partitions having the partition dimension from the first area or the second area, depending on a comparison of the first uncertainty and the second uncertainty.

2. The method according to claim 1, wherein:
the partitions having a partition dimension unequal to the maximum partition dimension are placed when the first uncertainty is less than the second uncertainty, and
the partitions unequal to the minimum partition dimension are placed when the first uncertainty is greater than or equal to the second uncertainty.

3. The method according to claim 1, wherein:
the partitions having a partition dimension from the first area are placed when the first uncertainty is less than the second uncertainty, and
the partitions having a partition dimension from the second area are placed when the first uncertainty is greater than or equal to the second uncertainty.

4. The method according to claim 1, wherein:
the partitions having the minimum partition dimension are placed when the first uncertainty is less than the second uncertainty, and
the partitions having the maximum partition dimension are placed when the first uncertainty is greater than or equal to the second uncertainty.

5. The method according to claim 1, wherein the partitions having a partition dimension from the first area, with the minimum partition dimension, are placed when it is not possible to measure a concentration of the analyte at a specified concentration value when the partitions having a partition dimension from the second area, having the second partition dimension, are placed.

6. The method according to claim 1, wherein:
the first uncertainty is determined using the first partition number of partitions having the minimum partition dimension, and
the second uncertainty is determined using the second partition number of partitions having the second partition dimension.

7. The method according to claim 6, wherein:
the first uncertainty and/or the second uncertainty are determined at a value from a maximum theoretical area for a measurement of the concentration of the analyte, and
the value corresponds to a logarithmic average of the maximum theoretical area.

8. The method according to claim 1, wherein:
the geometrical shape of the partitions is based on a wall, and
the wall at least partially delimits the partitions and has a specified wall thickness and/or wall height.

9. An analytical method for detecting an analyte in a microfluidic sample, comprising:
dividing the microfluidic sample, according to claim 1, into a plurality of partitions; and
examining at least one of the partitions for a presence of the analyte.

10. The analysis method according to claim 9, further comprising:
performing a digital PCR on at least some of the partitions.

11. A method for preparing a partition cohort of a microfluidic sample, wherein:
a partition surface is provided, and
the partition cohort of the microfluidic sample is performed according to a placement according to claim 1.
